(19) United States
(12) Patent Application Publication
(10) Pub. No.: US 2013/0030316 A1
(43) Pub. Date: Jan. 31, 2013

(54) METHOD AND DEVICE FOR MEASUREMENT OF EXHALED RESPIRATORY GAS TEMPERATURE

(71) Applicants: Todor Alexandrov Popov, Sofia (BG); Jaswinder Singh Gill, Singapore (SG)

(72) Inventors: Todor Alexandrov Popov, Sofia (BG); Jaswinder Singh Gill, Singapore (SG)

(73) Assignee: DELMEDICA INVESTMENTS LIMITED, ROAD TOWN, TORTOLA (VG)

(21) Appl. No.: 13/512,305

(22) PCT Filed: Nov. 29, 2010
(86) PCT No.: PCT/IB2010/055475

§ 371 Date: Oct. 16, 2012

(30) Foreign Application Priority Data
Nov. 30, 2009 (GB) 0920905.7

(51) Int. Cl. A61B 5/097; G01K 13/12

(52) U.S. Cl. 600/543

(57) ABSTRACT

An exhaled breath temperature (EBT) monitor that has a synchronized two-door shutter with movable individual doors coupled with a timer in order that only a pre-determined portion of the exhalation gas stream is selected for temperature monitoring. The doors can be either in a first position, whereby the shutter passes the portion of exhaled gas direct to atmosphere with no measurement of temperature, or in a second position, whereby the shutter passes the portion of exhaled gas to a chamber for measurement of temperature by a thermal core with a temperature sensor/reader.

### FIELD OF THE INVENTION

The present invention is concerned with a method and device for the measurement of the temperature of exhaled breath, such as may find application in medicine.

More specifically, the present invention is concerned with an apparatus useful in the performance of different medical investigations, including diagnostics and prevention and treatment of inflammatory lung and airway illnesses, such as diseases and allergies, in which assessment of the temperature of the exhaled breath may prove useful for the purpose of diagnosis and monitoring of the effect of anti-inflammatory treatments.

### BACKGROUND OF THE INVENTION

It is known that one of the most frequent socially important non-communicable diseases, asthma, is due to allergic inflammation of the airways. Evidence to this end has been collected by means of invasive methods of investigation: bronchoscopy with broncho-alveolar lavage and biopsies. Studies have established a quantitative relationship between the degree of inflammation of the airways and asthma severity, and also between a dose of an anti-inflammatory treatment and an ensuing clinical effect. Bronchoscopy is associated with an unpleasant experience for patients and also bears some risk, both during and after the investigation. Consequently, bronchoscopy cannot be applied routinely for the evaluation of airway inflammatory processes so as to tailor a therapy for an individual patient.

Noninvasive methods have been introduced as an alternative, for example, using examination of sputum, measurement of nitric oxide in exhaled air and assessment of mediators of inflammation in exhaled breath condensate. These approaches lack precision and consequently the results cannot reliably confirm a diagnosis or serve as a basis for treatment decisions. At the same time such non-invasive methods are time consuming and expensive. For example, measurement of nitric oxide in exhaled air, whose levels are higher in asthmatics, is complex, expensive and only suitable for use in specialized clinics.

Inflammation is a universal pathophysiological process and increased temperature is one of its five prominent features. In a patient with an inflamed airway, the inflamed airway mucosa acts to warm adjacent air to a higher level compared with the air adjacent to a comparative uninflamed mucosa. The extent of this warming of adjacent air depends upon the spread of an inflammatory region and on the level of inflammation.

Devices for the measurement of the exhaled breath temperature are described in (1) Piacentini G L, Bodini A, Zerman L, et al. ‘Relationship between exhaled air temperature and exhaled nitric oxide in childhood asthma’, Eur Respir J 2002; 20: 108-111; and (2) Paredi P, Kharitonov S A, Barnes P J. ‘Faster rise of exhaled breath temperature in asthma: a novel marker of airway inflammation?’ , Am J Respir Crit Care Med 2001; 165: 181-184.

International Patent publication No. 2007/012930 relates to an EBT (Exhaled Breath Temperature) monitor which provides measurement of the temperature of exhaled breath as a surrogate marker of the inflammation in the intrathoracic airways.

U.S. Pat. No. 3,613,665 describes an air monitor using readings from temperature sensors to initiate operation of the equipment, and is for single-breath sampling.

### OBJECTS OF THE PRESENT INVENTION

It is an object of the present invention to provide a method and device for measurement of the temperature of exhaled air from specific regions of the airway, which allows a high level of precision of the measurement, and which is simple and convenient to use by a patient or investigator.

Other objects of the present invention are to provide a device which is cost effective for routine application in medical practice, i.e. it has a minimum of moving parts and comprises as few component parts as practicable.

### SUMMARY OF THE INVENTION

The present invention provides an EBT monitor for the measurement of exhaled respiratory gas temperature during free voluntary tidal breathing, the monitor comprising:

an inlet port for receiving a stream of exhaled respiratory gas,

a housing defining a chamber, a temperature sensor located within the housing adapted for measuring the temperature of exhaled respiratory gas, pressure sensing means to detect the start of the exhalation operation,

valve means intermediate the air inlet and the housing to pass selectively part of the stream in to the chamber, and

time regulation means operable to initiate passage of the exhaled respiratory gas to the chamber at a pre-determined time interval after the start of the exhalation sequence.

In this way, a temperature measurement is made of only a section of the total stream of exhaled respiratory gas in one exhalation, and by appropriate selection of the timing of the valve operation the temperature of respiratory gas from any given part of the airway can be determined, allowing detection of what part of the airway may be inflamed or at an elevated temperature.

In a variant, the monitor may include, additionally or alternatively, volume regulation means to operate the valve means to pass a predetermined portion of the total volume of respiratory gas in an exhalation, and the volume regulation means may be operable to initiate passage of the exhaled respiratory gas to the chamber after a predetermined volume has entered the inlet port.

Other preferred features may include:
the temperature sensor comprises or is attached to a thermal reservoir of high heat capacity;
the thermal reservoir comprises a metal block;
the temperature sensor is a thermistor;
the monitor further comprises an electronic processor for processing electronic signals from temperature sensor and a display for displaying signals from the processor;
the housing serves to thermally insulate the chamber;
the housing comprises a Dewar flask.

As distinct from the apparatus of U.S. Pat. No 3,613,665, the present invention utilizes pressure to initiate the operation, because the system involves the measurement of temperature as the parameter for analysis. Another distinction is that the present invention involves analysis of multiple successive breaths to reach a thermal equilibrium. A further distinction of the present invention is that it allows sampling of air from a wide variety of parts and ranges of the airway, selectable in operation of the system, whereas U.S. Pat. No. 3,613,665 is limited solely to the alveolar section.

The present invention also provides a method of operating an EBT monitor for measuring exhaled respiratory gas temperature during free voluntary tidal breathing, the method comprising:

pressure sensing means detecting the start of the exhalation operation,

receiving a stream of exhaled respiratory gas at an inlet port, and

operating a time regulation means to initiate passage of the exhaled respiratory gas to the chamber at a pre-determined time interval after the start of the exhalation sequence by a valve means intermediate the air inlet and a housing, defining a chamber with a temperature sensor located within the housing adapted for measuring the temperature of exhaled respiratory gas, thereby to pass selectively part of the stream in to the chamber.

Advantageously, the method comprises operating the valve means to pass exhaled respiratory gas to the chamber for a portion of the total time interval for exhalation, and may comprise initiating passage of the exhaled respiratory gas to the chamber at a pre-determined time interval after the start of the exhalation sequence.

Additionally or alternatively, the monitor may comprise operating the valve means to pass a predetermined portion of the total volume of respiratory gas in an exhalation, and may comprise initiating passage of the exhaled respiratory gas to the chamber after a predetermined volume has entered the inlet port.

The present invention also provides a method for operating an EBT monitor for determining the temperature of exhaled respiratory gas during free voluntary tidal breathing, the method comprising the steps of:

i) providing a patient with an EBT monitor according to the present invention;
i) the patient exhaling into the inlet port of a said monitor;

ii) operating a valve means intermediate the air inlet and a housing, which defines a chamber with a temperature sensor located within the housing adapted for measuring the temperature of exhaled respiratory gas, thereby to pass selectively part of the stream in to the chamber; and
iii) recording said temperature reading.

In the present invention, the term thermal conductivity refers to thermal conductivity such as may be quantified in units of Wm <1>K<″1>. A material with a high thermal conductivity is a material having a thermal conductivity greater than that of polyethylene, such as metals, e.g. aluminum and copper. A preferred example is aluminum. Preferably, the thermal conductivity of the material forming the tube is at least 1, more preferably at least 10, even more preferably at least 40, most preferably at least 400200, Wm<−1>K<′1>at 23<0>C.

The apparatus of the present invention may comprise a thermistor or thermocouple as a temperature sensor. A thermistor is preferred. The temperature sensor may be electrically connected to an electronic processing unit and display for providing an indication to a user of the temperature of the respiratory gas inside the apparatus. The electronic processing unit preferably comprises means to indicate when thermal equilibrium has been reached, at which time an accurate temperature reading may be obtained.

In the present invention, the housing serves to thermally insulate the chamber from external atmospheric conditions. In one embodiment, the housing may comprise a Dewar flask. In another embodiment, the housing is formed from a plastics material having low thermal conductivity. Preferably, the thermal conductivity of the housing is no greater than 0.5, more preferably no greater than 0.1, even more preferably no greater than 0.05, most preferably no greater than 0.025, Wm<−1>K<″1>at 23<0>C. A thermally insulative housing assists the apparatus to reach temperature equilibrium during use and a more reliable measurement of breath temperature to be achieved.

In the present invention the term free voluntary tidal breathing refers to breathing which is not externally aided, such as by a respirator.

When measuring the temperature of the full exhalation cycle, the time required for the apparatus to reach equilibrium during use is preferably no more than 10 minutes, more preferably no more than 5 minutes, and may be less than 2 minutes.

The ability for a medical practitioner, skilled patient or other skilled user to determine small changes in exhaled breath temperature attributable to particular sections of the airways is seen to be potentially beneficial as a means of offering early control of inflammatory respiratory illness, which illnesses may be observed first by small but significant changes in exhaled breath temperatures which occur before the patient is observed to suffer acute symptoms of the illness.

Further, the present invention enables a patient to be treated with different medicaments (e.g. different active pharmaceuticals or different concentrations of the same active pharmaceuticals) depending upon the criticality of the treatment required as determined by the size of the difference between the measured exhaled breath temperature and the predetermined normal.

### APPLICATIONS OF THE PRESENT INVENTION

The present invention is directed to an EBT monitor which allows the measurement of temperature of a particular localized section of the airway. The EBT monitor of the present invention is able to selectively measure any one of a number of sections of the total airway from the lung, for example the central region or the peripheral region.

The present invention can be incorporated into an EBT monitor which provides temperature readings for the overall lung airway system, allowing comparison of EBT values measured by standard protocols (EBTst) with the EBT measured by a fractional protocol of the present invention (EBTfr), optionally for different regions of the airway system.

### ADVANTAGES OF THE PRESENT INVENTION

The present invention allows a ready, quick and easy temperature measurement of a variety of sections of the airway system, such sectional measurement and analysis not having been possible by conventional EBT monitors.

Furthermore, an operator of the EBT monitor, during the measurement procedure of an individual patient, can readily adjust the monitor settings to pinpoint a particular region of the airway for measurement and analysis.

### BRIEF DESCRIPTION OF THE DRAWINGS

In order that the invention may be more readily understood, a description is now given, by way of example only, reference being made to various embodiments of the present invention, in which:

FIG. 1 is a cross-section of a first embodiment of an EBT monitor in accordance with the present invention;

FIGS. 2 and 3 are schematic drawings of a second embodiment of an ABT monitor of the present invention in two operating modes;

FIG. 4 is an illustration of the breathing sequence and shutter operation of the monitor of FIGS. 2 and 3;

FIG. 5 is a diagram of the lung airways system indicating particular areas of relevance to the present invention;

FIG. 6 is a third embodiment of an EBT monitor system embodying the present invention; and

FIGS. 7 and 8 are print-outs of a patient's response to the EBT system of FIG. 6.

### DETAILED DESCRIPTION OF THE PRESENT INVENTION

A standard protocol for exhaled air temperature measurement during free voluntary tidal breathing used in conventional EBT monitors is as follows. The subject inhales through the nose room air which is preferably within the temperature range 18[deg.]-22[deg.]C and then exhales into the air inlet of the device. Measuring the resistance of a thermal sensor assesses the temperature in the thermal chamber. It is increased by the cumulative effect of multiple exhalations on the part of the examined subject. The measurement continues until temperature equilibrium is established within this closed system between the temperature of the exhaled air and the temperature of the metal core (or block) inside the thermal chamber.

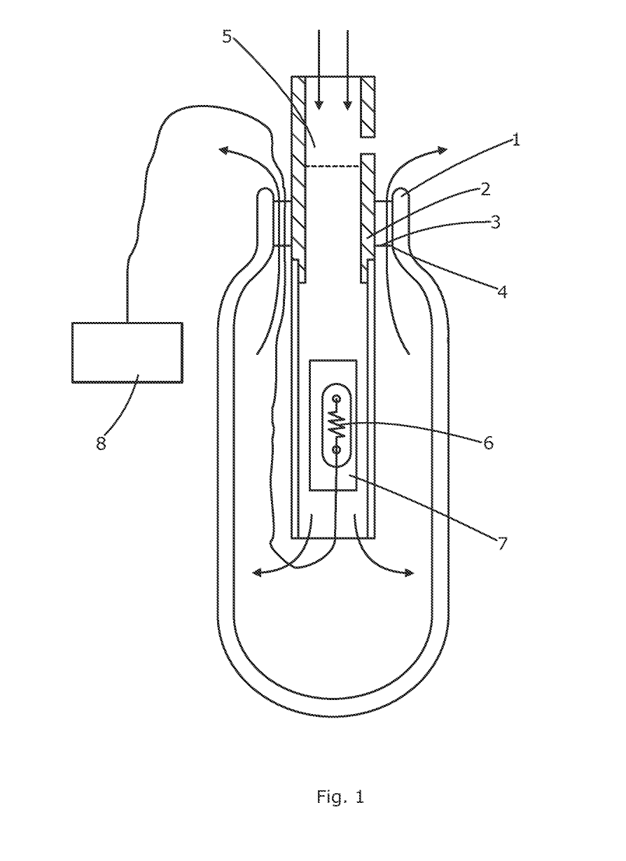

The method of the present invention may be performed using an EBT monitor of the present invention as shown in FIG. 1 which incorporates a thermo-isolated bottle-like vessel, or housing, with a mouthpiece at its opening, with a heat accumulating metal core with a thermal sensor attached thereto and a valve allowing selective measurement of part of the airway system.

In one embodiment of the monitor, the vessel represents the housing of a thermally insulated chamber constructed as a Dewar flask, also termed a vacuum flask, with a mouthpiece positioned into an in-going tube situated in an, in use, upper part of the chamber. The tube is fixed to the mouth of the Dewar vessel by means of an element with outlets. In the interior of the tube from top to bottom are situated a valve and temperature sensor, mounted on a metal core; the sensor is linked to a reading device outside the chamber. The number of outlets could be changed so as to allow appropriate resistance to optimally modulate the rate of breathing.

The EBT monitor of the present invention features simplified and robust construction. It allows subjects to measure the temperature of their exhaled air from a section of the airway system in an easy and convenient way. The thermally isolated Dewar vessel enables the incoming exhaled air to impart its thermal energy to the high thermal capacity metal core and the attached thermal sensor within the time frame until the next exhalation. When measuring the temperature of the overall airway system, adding up of subsequent exhalations allows achievement of a thermal balance mostly independent of the ambient conditions and reflective of the integral temperature within the lungs. A chance interruption of the breathing rhythm (swallowing or pausing on the part of the subjects) does not bear any significant effect on the final result. The EBT monitor could be applied as individual gauge for subjects with airway inflammatory disease to monitor the course of their disease and to help timely modifications of the treatment scheme and prevent exacerbations.

The EBT monitor of the present invention may comprise a processor to automatically monitor the progress of the measurement, to produce an indication, such as sound, when thermal equilibrium is reached and to present the measured temperature on a display on an external surface or body of the device.

The EBT monitor of the present invention could be used in practice to give the user initial information about the exhaled breath temperature of the entire airway system of a given patient with inflammatory airway disease together with EBT values for one or more specific regions of the airway system, to be further used as reference in the course of treatment. The EBT monitor of the present invention may be operated by a patient or by medical personnel, and may also comprise storage facilities for recording measurements for a given patient taken on a regular basis, such as day-to-day, to be stored in a database to help the doctor or the patient take a needed action.

An example of the method to measure exhaled breath temperature for the entire airway system involves at least 5 sequential assessments. The patient breathes out through a mouthpiece into a thermal chamber, whose temperature is determined by the readings of the electrical resistance of a temperature sensor, and can be seen on a display. The values keep changing until the temperature in the thermal chamber reaches equilibrium with the temperature of the air exhaled by the subject. When such a plateau is reached, the corresponding temperature is marked as the temperature of the exhaled breath of the tested subject.

The method of the invention is made possible by use of an EBT monitor built to this purpose. FIG. 1 represents a schematic drawing of an exemplary embodiment of the EBT monitor for exhaled respiratory gas from only a section of the airway system. The EBT monitor comprises a thermal chamber or housing made as a Dewar vessel 1, with an inlet tube 2 in its upper part fixed by means of a tightening or securing element 3 with in-built air outlets orifices 4. It is preferable that the thermal chamber has an elongated cylindrical shape softly folding to form its bottom part with the shape of a hemisphere. This shape will ensure smooth flow of the exhaled air and avoid turbulences. The number of outlet holes may vary to achieve optimal aerodynamic resistance favoring the desired breathing rate.

In the interior of the air inlet tube 2 from top to bottom are situated a two-position shutter valve 5 and temperature sensor 6, mounted on a metal core 7; the sensor is linked to a reading device 8 situated outside the chamber 1. The temperature sensor is calibrated in two points within the range 0[deg.]-36[deg.]C. The temperature of the thermal chamber is assessed by determining the electrical resistance (Ohms) of the temperature sensor, which is in the form of a thermistor.

In operation of the present invention, two sample inhalation/exhalation cycles are conducted, in order to provide the monitor with two examples of a dynamic sequence and timing of the overall exhalation cycle, for analysis and calculation of the required timing and/or volume actuation in order to select the appropriate section of the airway system.

After a rest, e.g. of about 5 minutes, to allow the EBT monitor to cool down, the patient exhales again into the mouthpiece which has a pressure sensor to initiate the operation when appropriate but this time, by appropriate actuation of valve 5, the monitor diverts some of the stream such that only part of the exhaled air enters the chamber, the remainder passing directly to atmosphere.

The valve 5 has one position in which it allows exhaled respiratory gas from the mouthpiece to enter the thermal chamber 1 (when the total airway readings are being performed, and while the selective airway readings are being done), and another position in which it directs exhaled respiratory gas direct to atmosphere (when measurement of the immediate temperature is not required).

When operating the EBT monitor for measuring the temperature of the entire airway system, the investigator recorded the resistance values at one-minute intervals and discontinued the measurement when the last registered value was the same as the preceding one. The time for achieving thermal equilibrium was within the range of 5 to 12 minutes.

In the EBT monitor, a thermal reservoir is heated by exhaled air. Each subsequent exhalation increases the inside temperature until an equilibrium is reached. As the reservoir has a relatively high volume, minimal and short temperature changes (artifacts) do not affect significantly the end result. It is recommended that the measurement is made at room temperature (18[deg.]-22[deg.]C), which ensures reproducible and reliable results. The EBT monitor is made ready for measurement by placing a mouthpiece in the inlet tube. The subject holds it with one or both hands, breathes in through the nose and breathes out through the mouthpiece at a rate he or she finds comfortable. He or she is advised against hyperventilation and is allowed to make short pauses to swallow or make verbal comment. The investigator marks the readings of the ohmmeter in a report form at minute intervals until a value repeats itself. Then the value is converted to the corresponding temperature following a formula worked out when initial calibration of the device was made. When the measurement is complete, the EBT monitor is dissembled, cleaned with cool water, dried and assembled for the next measurement.

An EBT monitor of the present invention is preferably provided with a disposable mouth piece for insertion into or placing over a mouth of a patient. A disposable mouth piece is preferable on hygiene grounds. The present invention, by warming an incoming airway, may also warm a mouth piece portion of an incoming airway. As this warming is achieved by redirected backflowed air from the monitor of the invention, the present invention realizes a convenient method of warming a mouthpiece entrance without providing any additional ducting or separate heating element. As described above, the heating of an incoming airway with backflowed air provides a more reliable measurement of breath temperature, especially when combined with a device portion of high heat capacity in proximity to the temperature sensor.

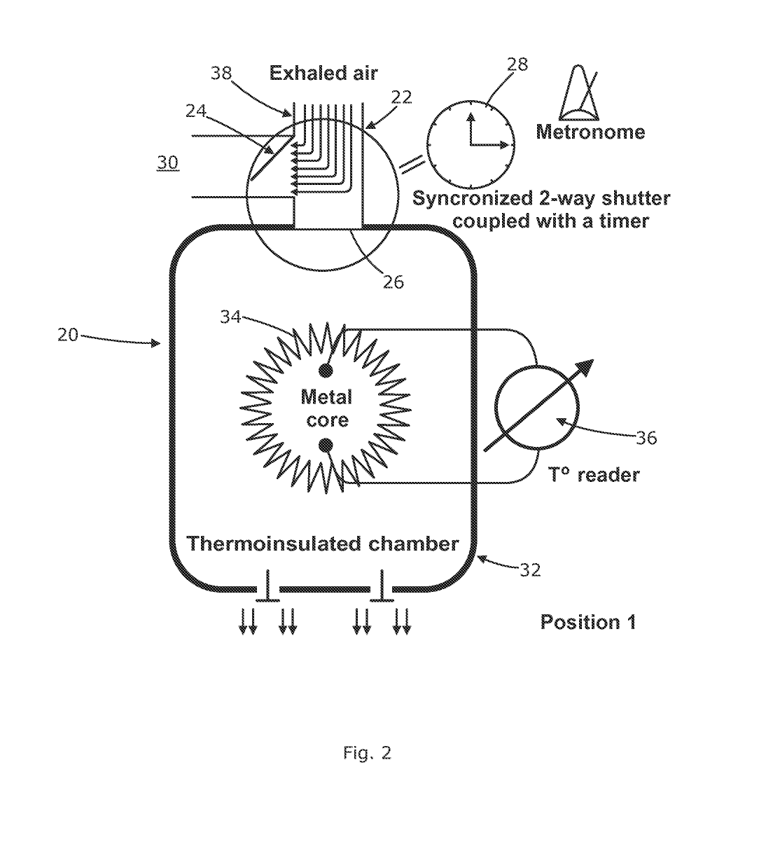

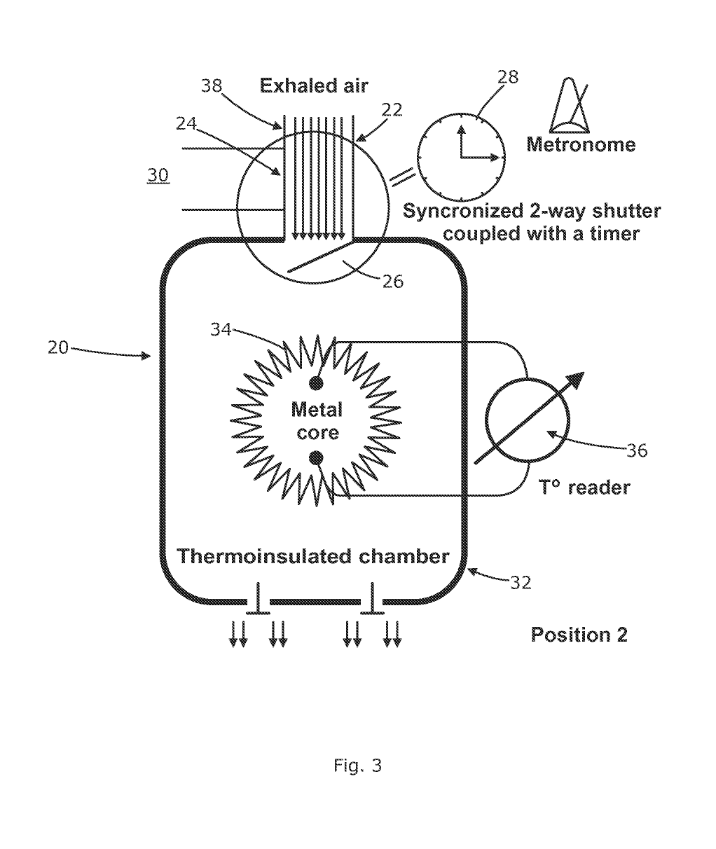

There is shown in FIGS. 2 and 3 a second embodiment of EBT monitor 20 having a synchronised two-door shutter 22 with movable individual doors 24, 26 coupled with a timer or adjustable clock 28 (in a variant, this may be achieved by a time delay operating as from the start of the exhalation operation) in order that only a pre-determined portion of the exhalation gas stream is selected for temperature monitoring. Thus the doors 24, 26 can be either in position 1 (FIG. 2) whereby the shutter 22 passes that portion of exhaled gas direct to atmosphere 30 with no measurement of temperature, or in position 2 (FIG. 3) whereby the shutter 22 passes that portion of exhaled gas to chamber 32 for measurement of temperature by thermal core 34 with temperature sensor/reader 36.

The start of the exhalation operation or cycle is detected by a pressure sensor 38 which is located at the inlet of the opening of the valve system immediately after the patient mouthpiece. It is capable of sensing pressures in the range 50 to 150 cmH₂O, which covers both genders, adults and children.

Clearly, when a temperature measurement of the entire exhalation is required, the doors 24, 26 of shutter remain in position 2 the entire cycle.

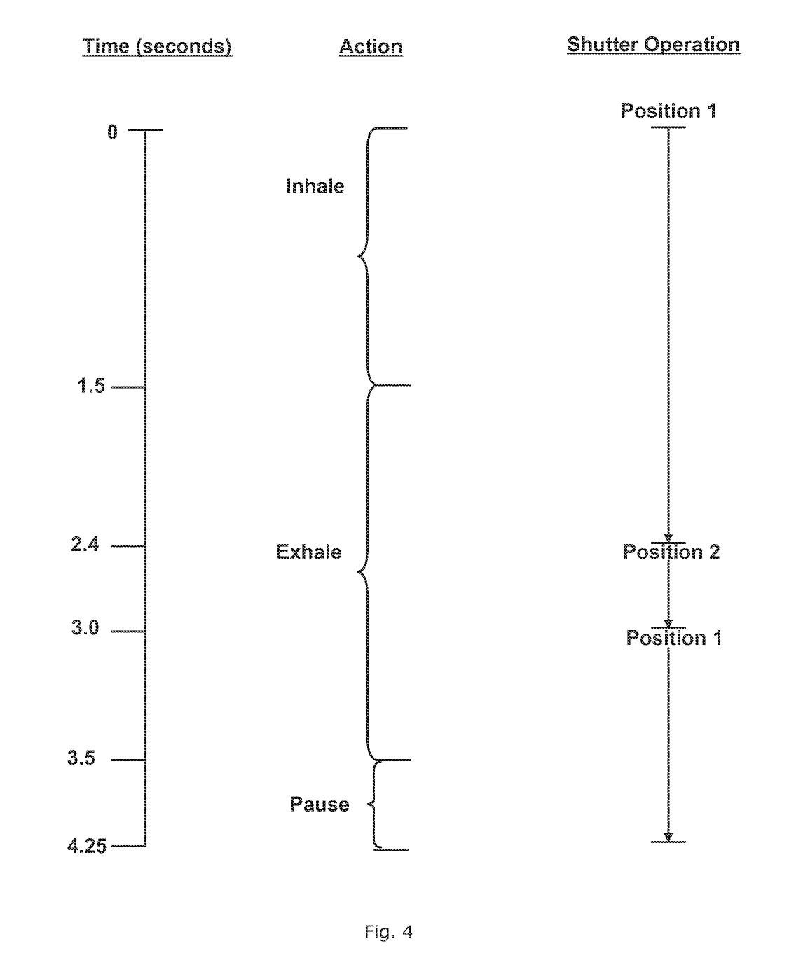

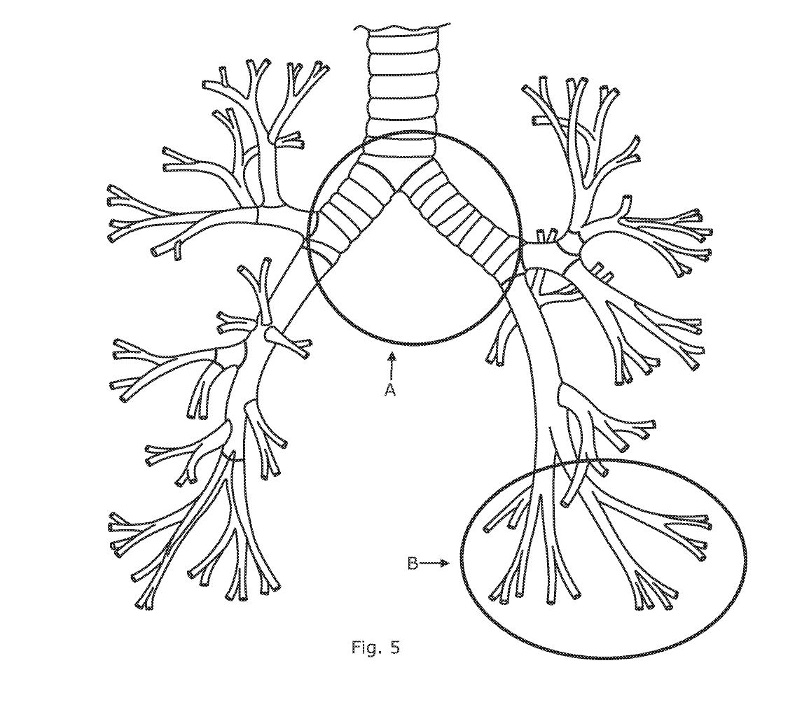

FIG. 4 shows schematically the corresponding time sequences, actions in the inhalation/exhalation cycle and shutter 22 operation for the temperature measurement in the central airways region of a typical male patient aged 35 of height 1.75 metres with a lung capacity of 4.5 metres and an inhalation/exhalation cycle of 10 per minute, when using the EBT monitor 20. Such temperature measurement would be done in order to look for inflammation in the central section of the airway system as shown by the circle A in FIG. 5, as typically evident in smokers.

When looking for inflammation typical in asthmatics with small airway involvement (usually the difficult-to treat patients), there would be operation of shutter 22 towards the end of the exhalation period, in order to measure temperature of the gas coming from the peripheral airways region as shown by loop B in FIG. 5.

The EBT monitors of the present invention may incorporate pre-determined settings and/or adjustable controls for positioning of the shutter 22 and doors 24, 26 in order to ensure appropriate temperature measurement in all relevant zones of the airway for all individuals in all circumstances.

There may be provided, just down-stream of the inlet port of EBT monitor 20, one or more sensors to monitor any one or more of the following parameters of the exhaled respiratory gas stream as it enters the monitor, namely: flow rate, speed, volume, constituents and humidity. By dynamically monitoring the values of any or all of these parameters, accurate and precise positioning of the shutter doors 24, 26 within the appropriate part of the exhalation cycle to correspond to the required parts of the airway for temperature measurement and analysis can be optimized.

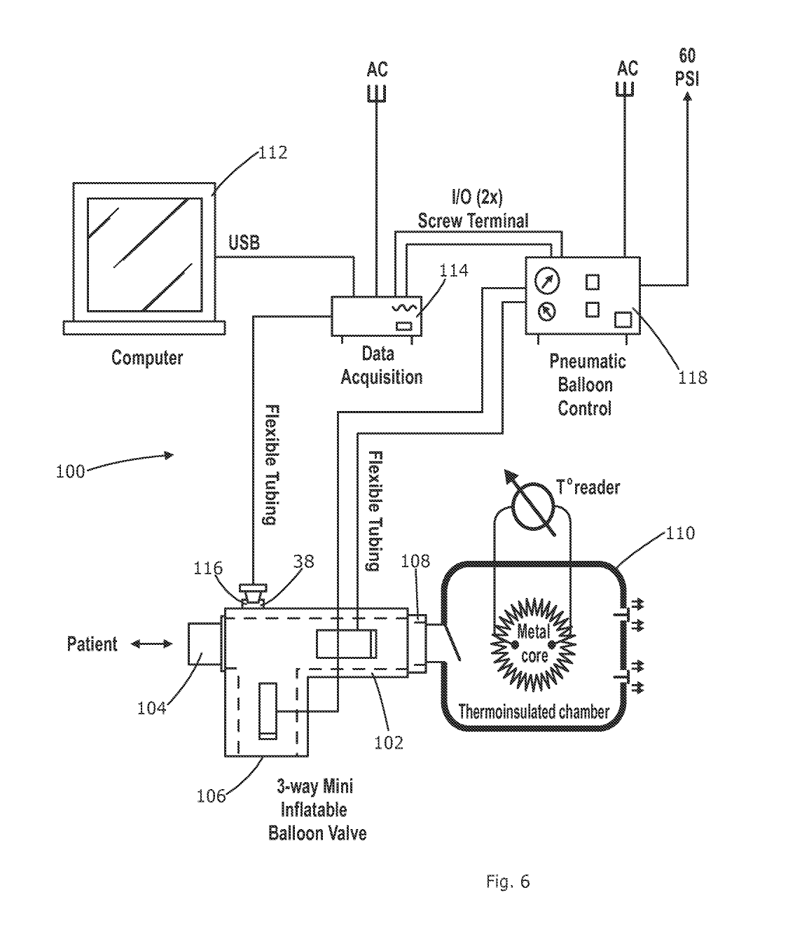

FIG. 6 is a schematic diagram of an EBT monitor 100 of the present invention, and including a three-way inflatable balloon valve 102 with a mouthpiece 104 into which a patient blows when appropriate. The valve unit has one output 106 which passes exhaled air to atmosphere, and another outlet 108 which passes exhaled air to the temperature measurement unit 110.

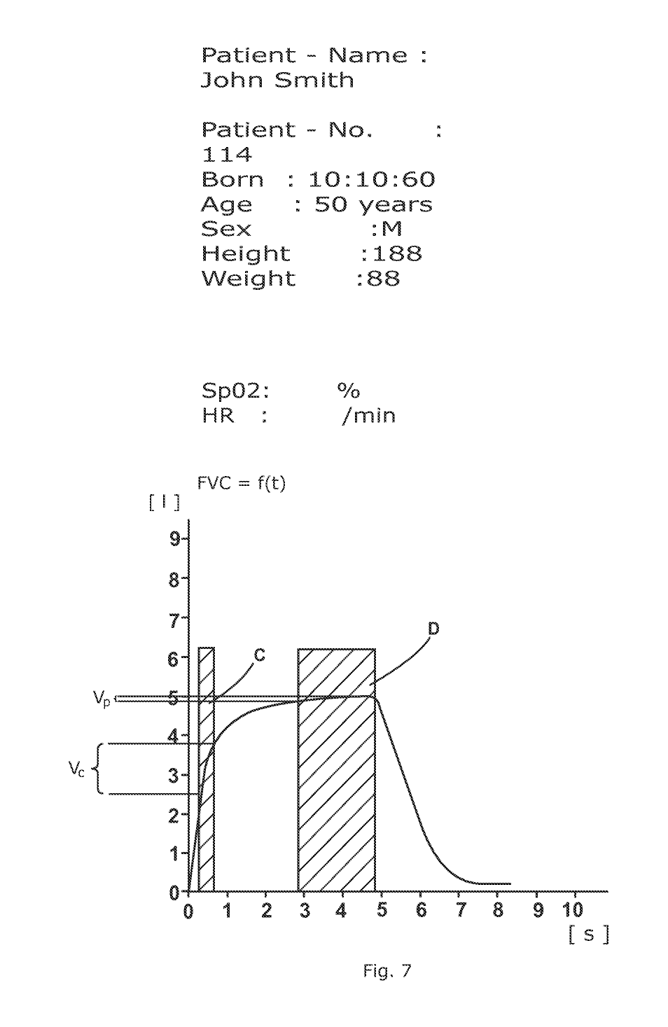

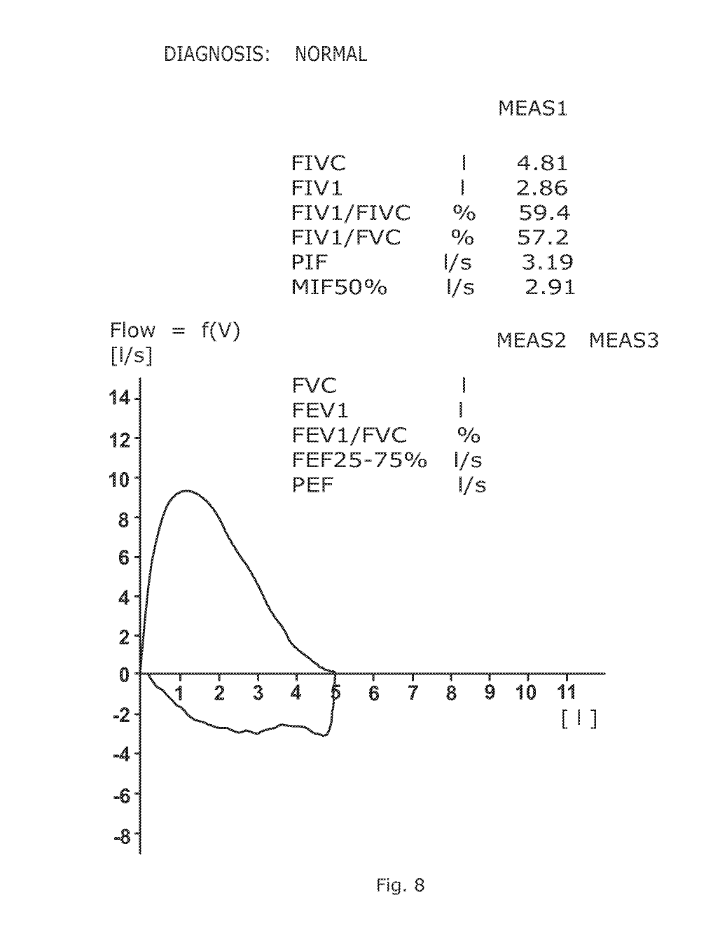

EBT monitor 100 has a processor unit 112 with software providing visualization of the temperature measurement and the operation of the valve system on its screen. Data acquisition unit DAQ 114 is operable to receive input signal from the pressure sensor 38 located in a nested pressure sensor 116 situated immediately after the mouthpiece 104 into which the patient has started an expiration. This triggers the operation of the valve system as follows:
1) DAQ signals the adjustable timer of the Pneumatic Balloon Control 118 to begin the preset sequence of events steering the flow of exhaled air in and away from the thermal chamber of the EBT measuring device 110.
2) The time span of entry of exhaled air into the EBT measuring device 110 is adjusted to correspond to a fraction of the whole duration of the respiration cycle, determined on the basis of the individual flow-volume and time volume curves of a given patient. [FIGS. 7 and 8 show printouts of a spirometry, indicating time on the abscissa for allowing into the device a predetermined volume of air (see shaded areas—central, then peripheral airways)].
3) After the airflow into the EBT device is interrupted by the valve system, the cycle is terminated, and the DAQ unit 114 switches to stand-by mode and is triggered again by the next signal from the pressure sensor.
4) When the temperature of the EBT measuring device 110 reaches a plateau under the input of cumulative portions of air deriving from the identical segments of the respiration cycle, the measurement comes to an end.

As the measurement requires multiple successive breaths, patients are trained to breathe uniformly to the beat of a metronome, individually adjusted to a full vital capacity inspiration/expiration manoeuvre.

As the measured exhaled breath temperature requires ultimate precision, any electro-mechanical influences may introduce bias. Therefore, the present invention incorporates a pneumatic balloon system operating the valves, the volume of the valve balloons and the associated changes of the gas in them being negligible.

FIGS. 7 and 8 are print-outs of a patient's responses to an EBT monitor system of the present invention serving as a basis for defining/calculating the timeframe of operation of the valve system to suit the individual subject's characteristics and the airway segment to be assessed by the EBT monitor system of FIG. 6.

FIG. 7 shows the volume-against-time graph, for selection of volumes (in blue) representative of different segments of the airways, for calculation of the time (in red) for the valve operation. Shaded area C is the time(s) representative of the airflow “rinsing” the central airways; shaded area D is the time(s) representative of the airflow deriving from the peripheral airways, the central airways. The volume on the Volume/Time graph of FIG. 7 is selected, from which the time points are calculated and automatically used to instruct the timer of the valve system.

FIG. 8 shows the flow-volume loop, and is used for automated selecting and adjusting of the timer periods required.

The graph relationship and analysis is used for automation of the procedure of search of airway segments most affected by inflammation of individual patients, and is used to effect automated setting/selecting/adjusting of the time intervals for example in a first one or more “setting” cycles before the sequence of 5 cycles to achieve temperature equilibrium for the measurement operation. In a variant, the microprocessor of the EBT-measuring unit 110 repeatedly checks and, when the plateau is reached, it signals the end of the measurement.

1. An exhaled breath temperature (EBT) monitor for the measurement of exhaled respiratory gas temperature during voluntary tidal breathing, the monitor comprising:
an inlet port for receiving a stream of exhaled respiratory gas,
a housing defining a chamber,
a temperature sensor located within the housing adapted for measuring the temperature of exhaled respiratory gas,
a pressure sensor to detect the start of the exhalation operation,
a valve intermediate between the inlet port and the housing to selectively pass part of the stream in to the chamber, and
a time regulator operable to initiate passage of the exhaled respiratory gas to the chamber at a pre-determined time interval after the start of an exhalation sequence for a plurality of streams of exhaled respiratory gas, each corresponding to a specific region of the airway system.

2. The monitor as claimed in claim 1 wherein the time regulator comprises an adjustable clock.

3. The monitor as claimed in claim 1 wherein the monitor further comprises a volume regulator to operate the valve to pass a predetermined portion of a total volume of respiratory gas in an exhalation.

4. The monitor as claimed in claim 1, wherein the volume regulator is operable to initiate passage of the exhaled respiratory gas to the chamber after a predetermined volume has entered the inlet port.

5. The monitor as claimed in claim 1, wherein the temperature sensor comprises or is attached to a thermal reservoir of high heat capacity.

6. (canceled)

7. (canceled)

8. The monitor as claimed in claim 1, wherein the monitor further comprises an electronic processor for processing electronic signals from the temperature sensor and a display for displaying signals from the processor.

9. The monitor as claimed in claim 1, wherein the housing serves to thermally insulate the chamber.

10. (canceled)

11. A method of operating an exhaled breath temperature (EBT) monitor for measuring exhaled respiratory gas temperature during voluntary tidal breathing, the method comprising:
measuring pressure detected at the start of exhalation,
receiving a stream of exhaled respiratory gas at an inlet port, and
operating a time regulator for a plurality of exhaled respiratory gas, each corresponding to a specific region of the airway system to initiate passage of the exhaled respiratory gas to a chamber at a pre-determined time interval after the start of an exhalation sequence by a valve intermediate between the inlet port and a housing, wherein the housing defines the chamber with a temperature sensor located within the housing adapted for measuring the temperature of exhaled respiratory gas, and
selectively passing part of the stream in to the chamber.

12. The method as claimed in claim 11 comprising operating the valve to pass a predetermined portion of the total volume of respiratory gas in an exhalation.

13. The method as claimed in claim 12 comprising initiating passage of the exhaled respiratory gas to the chamber after a predetermined volume has entered the inlet port.

14. The monitor according to claim 1 operable for comparison of measurements for different regions of the airway system.

15. The monitor according to claim 1 operable for search and/or analysis of airway segments.

16. The monitor according to claim 1, wherein the time regulator is operable to initiate passage of exhaled respiratory gas (i) for the central section of the airway system, and (ii) for the peripheral section of the airway system.

17. A monitor according to claim 1 comprising a processor operable to handle multiple successive breath sequences of a patient.

18. A monitor according to claim 1 operable to monitor for a plateau of temperature measurement.

19. The method according to claim 11 further comprising comparison of measurements for different regions of the airway system.

20. The method according to claim 11 further comprising search and/or analysis of airway segments.

21. The method according to claim 11 comprising operating the time regulator to initiate passage of exhaled respiratory gas (i) for the central section of the airway system, and (ii) for the peripheral section of the airway system.

22. The method according to claim 11 comprising repeating the method with one or more successive breath sequences of a patient.

23. A method according to claim 11 comprising monitoring for a plateau of temperature measurement.
